(19) United States
(12) Patent Application Publication
(10) Pub. No.: US 2005/0113854 A1
(43) Pub. Date: May 26, 2005

(54) Cervical conization device

(76) Inventor: John E. Uckele, Rochester Hills, MI (US)

Correspondence Address: Myers Dawes Andras & Sherman LLP;Susite 1150, 19900 MacArthur Blvd., Irvine, CA, 92612, US

(21) Appl. No.: 10/998,291

(22) Filed: Nov. 26, 2004

Related U.S. Application Data
(60) Provisional application No. 60/525,556, filed on Nov. 25, 2003.

(51) Int. Cl. A61B 17/32

(52) U.S. Cl. 606/167; 606/170; 606/193

(57) ABSTRACT

A surgical instrument for removal of a conical section the cervix for pathological examination is disclosed. The device includes a circular knife having a plurality of double-edged blades, the edges of adjacent blades enclosing against one another when the device is operated from unengaged to engaged positions. A hollow plunger is further disclosed used to actuate the circular knife. Novel pivot devices are additionally included to provide translational movement of the hollow plunger bar. A pronged stabilization rod is disclosed to prevent the circular knife from moving away from the target tissue during a cutting stroke. The device is an improvement over prior devices and procedures in that it provides a precision conical tissue sample ideal for analysis with minimal resulting bleeding.

This application claims the benefit of provisional patent application No. 60/525,556, filed on Nov. 25, 2003.

### BACKGROUND OF THE INVENTION

1. Field of the Invention

The present invention pertains generally to medical instruments. More particularly, the present invention relates to surgical instruments for performing a uterine cervical conization. The present invention is particularly, but not exclusively, useful as a surgical instrument for removal of a conical section of cervical tissue for a biopsy.

2. Description of the Prior Art

Conization of the cervix, or “cone” biopsies are known for diagnosis, prevention, and treatment for cancer of the uterine cervix. Such cervical cancer usually progresses slowly over an extended period from the first appearance of pre-cancerous abnormalities. With today's sophisticated screening programs involving regular uterine pap smear tests, colposcopy and other procedures, it is increasingly likely to detect certain abnormalities in their early stages and successfully treat these patients and preserve reproductive functions. Precancerous, abnormal cells present on cervical tissue is a condition known as dysplasia or cervical intraepithelial neoplasia (CIN).

Cervical conization is a procedure performed to remove abnormal tissue from the cervix to further diagnose cancer and to better understand results of a pap smear and/or colposcopy. The procedure may further be used to treat chronic cervicitis (inflammation of the cervix) and it is sometime called a “cone” biopsy because a cone-shaped wedge of tissue is removed from the cervix and examined under a microscope. The cervix is located in the lower part of the uterus that is high in the cervical canal, making access relatively difficult, wherein the uterus is the muscular organ at the top of the vagina where menstruation begins and babies gestate.

Technological developments have introduced a wide variety of surgical procedures for conization of the cervix. Among them are the traditional cold scalpel procedure, punch biopsy, and loop electrosurgical excision procedure (LEEP). The electrosurgical procedure, in particular, is problematic in that a fragmented sample may be obtained that does not have a “cone shape” as is desired for pathologic examination. Also, any lateral movement of the loop device may be damaging to the lateral vaginal wall.

Additionally in the art are manual tool-based procedures such as the punch biopsy. Exemplary devices in the prior art are essentially modified biopsy forceps in that they hinge about a pivot point and close around the target tissue. These devices generally require excessive “sawing,” “grabbing,” or rotational cutting that may damage tissue more excessively than desired and produce a relatively high amount of bleeding adding complications. Further, as with the LEEP procedure, the quality of the sample and the resulting bleeding is highly dependent upon the skill and experience of the surgeon and may require the surgeon to estimate the distance and angle of the cut.

Accordingly, it is an object of present invention to provide a medical device for performing uterine cervical conization that yields high quality tissue samples for analysis with a minimum degree of surgical skill and estimation. It is further an object of the present invention to provide a surgical instrument for excising a tissue sample that results in a minimum amount of bleeding. Additional objects and advantages of the present invention will be apparent from the following invention summary and description.

The invention may also be regarded as a surgical instrument for excising a tissue sample from a tissue mass, the surgical instrument comprising: a stabilization rod having a distal and a proximal end and defining a longitudinal axis; a hollow plunger bar disposed about the stabilization rod having a distal and proximal end; a circular knife having a plurality of blades converging on a point of convergence, the circular knife disposed about the distal end of the hollow plunger bar, the circular knife further having engaged and unengaged positions; and a housing having a distal portion accommodating the circular knife, the housing distal portion having an annulus tapered at an end thereof, the tapered annulus directing the circular knife toward the point of convergence.

Lastly, the invention may be regarded as a surgical instrument for excising a tissue sample from a tissue mass, the surgical instrument comprising: a hollow plunger bar disposed about the stabilization rod having a distal and proximal end; and a circular pentagonal knife having five blades converging on a point of convergence, the circular knife disposed about the distal end of the hollow plunger bar, the circular knife further having engaged and unengaged positions.

### BRIEF SUMMARY OF THE INVENTION

The present invention specifically addresses and alleviates the above mentioned deficiencies associated with the prior art. More particularly, the present invention, in a preferred embodiment is, a surgical instrument for excising a tissue sample from a tissue mass, the surgical instrument comprising: a hollow plunger bar defining a longitudinal axis having a distal and proximal end; a circular knife having a plurality of blades converging on a point of convergence, the circular knife disposed about the distal end of the hollow plunger bar, the circular knife further having engaged and unengaged positions; and a housing having a distal portion accommodating the circular knife, the housing distal portion having an annulus tapered at an end thereof, the tapered annulus directing the circular knife toward the point of convergence.

While the apparatus and method has or will be described for the sake of grammatical fluidity with functional explanations, it is to be expressly understood that the claims, unless expressly formulated under 35 USC §112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC §112 are to be accorded full statutory equivalents under 35 USC §112. The invention can be better visualized by turning now to the following drawings wherein like elements are referenced by like numerals.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a perspective view of a cervical conization device incorporating the presently preferred embodiment of the present invention;

FIG. 1B is a perspective view of the illustration in FIG. 1A with major portions of the device housing cut away;

FIGS. 2A and 2B illustrate cross-sectional views of the preferred embodiment of the present invention taken along sectional line 2-2 in FIG. 1A;

FIGS. 3A through 3C illustrate a preferred stabilization rod of the present invention inner and outer sleeves;

FIGS. 4A through 4C are side and top plane views of a hollow plunger of the present invention;

FIG. 5. is an end view of the presently preferred embodiment of the present invention;

FIGS. 6A and 6B illustrate a preferred circular knife of the present invention;

FIG. 6C illustrates a flat cut-out of the preferred circular knife of the present invention;

FIG. 7 is a cross-sectional view of a second preferred embodiment that uses a tri-blade system where the blades 18a move radially in and out within corresponding guide notches in an annulus 52′;

FIG. 8A is an exploded perspective view of a portion of the second preferred embodiment shown in FIG. 7, showing how a guide tab 118 on the bottom of one of the three blades 18a′ engages the guide notch 152 in the annulus 52′, a spring 153 being present to bias the blade 18a′ outward;

FIG. 8B shows the position of the three blades 18a′ when the annulus 52′ in the assembly suggested by FIGS. 7 and 8A is moved forward and the blades are converged;

FIG. 9A is a perspective view of the second preferred blade assembly 18′;

FIG. 9B is an end view of the second preferred blade assembly 18′; and

FIG. 9C is a perspective view of the second preferred blade assembly with resistive pads 161, conductors 162, and a wire bundle 163 for implementing an electro-cauterization process on the exterior of the second preferred blade assembly 18′.

### DETAILED DESCRIPTION OF THE PREFERRED EMBODIMENTS

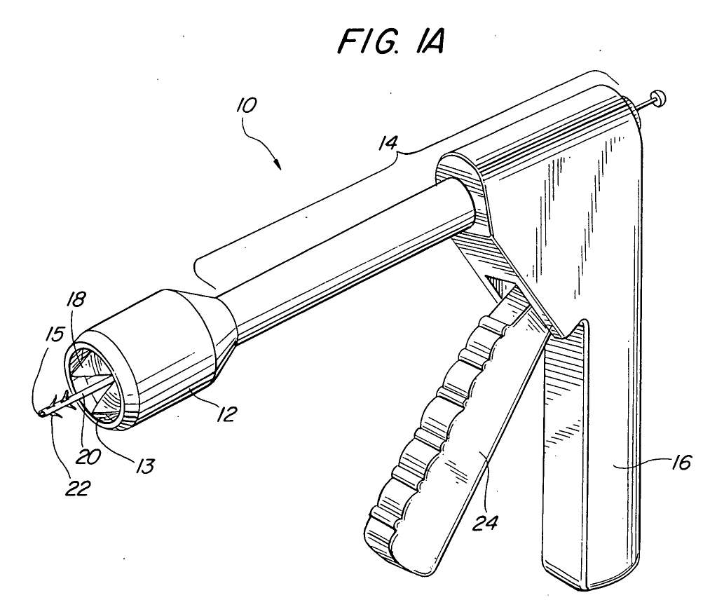

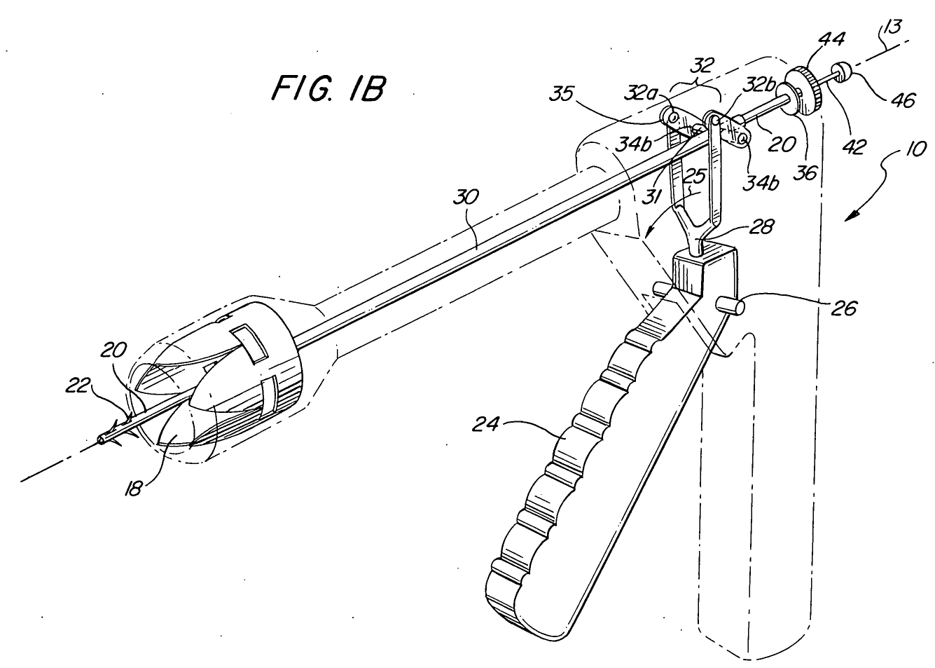

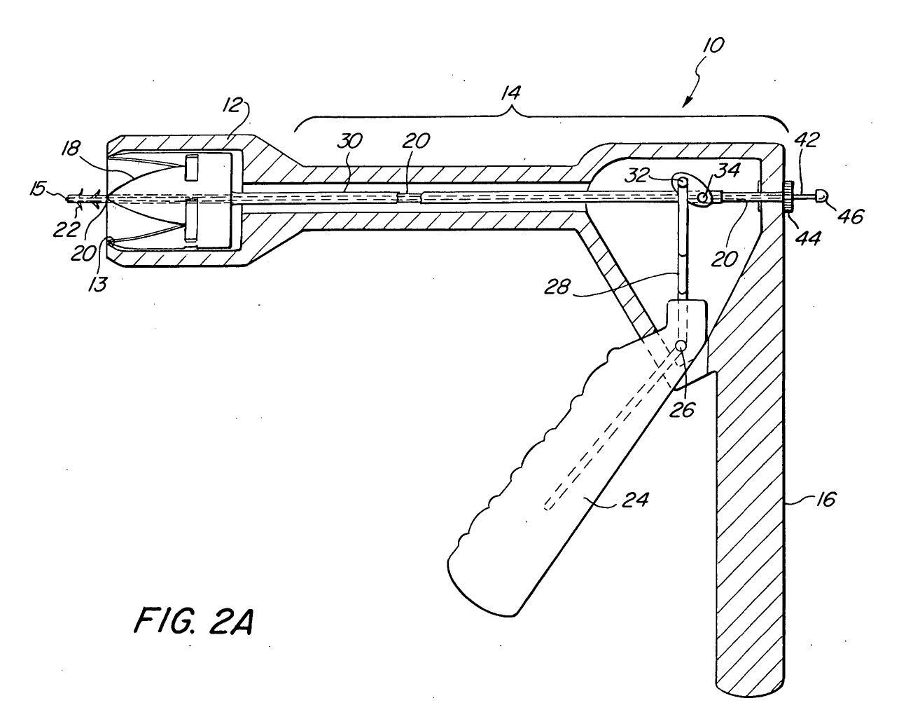

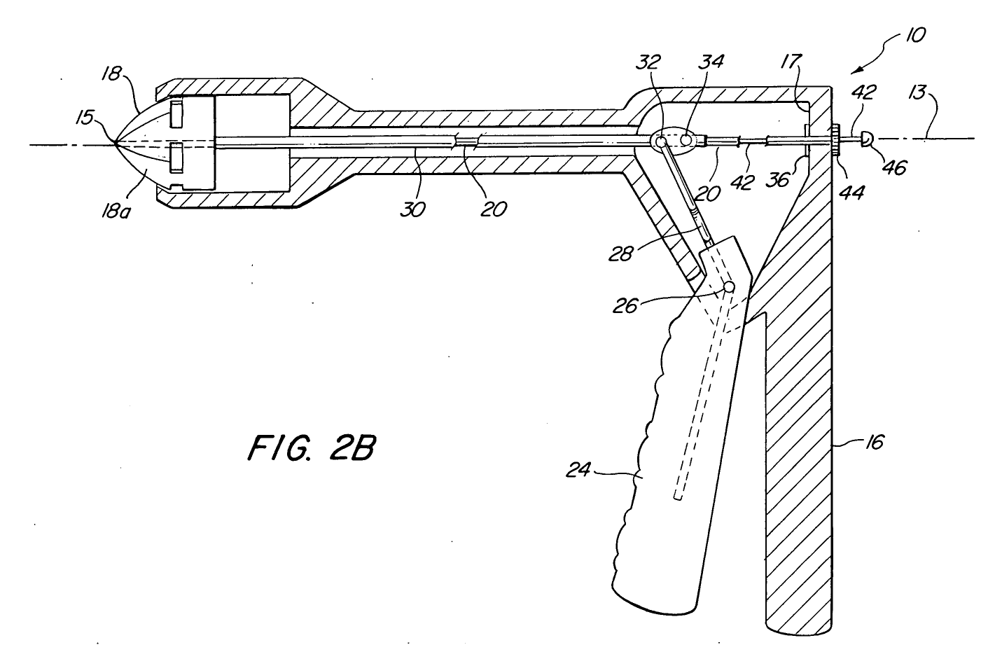

Referring initially to FIG. 1A, a perspective view of a preferred embodiment of a cervical conization device is illustrated and generally designated 10. As shown, device 10 has a housing having a distal portion 12 that houses a plurality of substantially pentagonal blades 18a (FIG. 6C) that collectively form a circular knife 18 of the present invention. The housing further has an elongated sleeve portion 14 that houses the actuating devices of the invention as discussed herein in further detail. A housing stationary handle portion 16 is opposite a trigger handle 24 and is generally perpendicular to the housing elongated sleeve portion 14 of device 10. As further described herein, blades 18a (FIG. 6C) precisely converge on a point 15 just beyond a plurality of barbs 22 of a barbed stabilization rod 20 of the present invention. Whereas exemplary prior art devices converge on a pin, by comparison, the present invention converges past the stabilization rod 20 and therefore is enveloped (as shown in FIG. 2B) and ensures a cleaner cut.

Referring to FIG. 1B, a perspective view of the illustration in FIG. 1A is shown with portions of the housing drawn in transparency to show the actuating devices of the present invention. Hollow plunger 30 is employed to provide translational movement of the circular knife 18. As shown, the circular knife 18 is a circular pentagonal knife 18 in the preferred embodiment. The translational movement is provided by first squeezing trigger handle 24 to cause rotational movement 25 of Y-shaped swing arm 28 about fulcrum pin 26. A first pivot device 32 in conjunction with second pivot device 34 is provided to convert rotational movement 25 to translational movement of the hollow plunger bar 30. A tee portion 31 of hollow plunger bar 30 has first and second ends and is provided to connect to the second pivot device 34, first and second pins 34a, 34b to middle segments 35. Further, middle segments 35 are connected to Y-shaped plunger bar 28 via first pivot device 32 first and second pins 32a, 32b. Stabilization rod 20 is illustrated having barbs 22 on a distal end with respect to an instrument 10 user (doctor). Proximally, stabilization rod 20 has a seat 36 to secure the stabilization rod 20 against a housing rear inside wall 17, as further illustrated in FIGS. 2A and 2B.

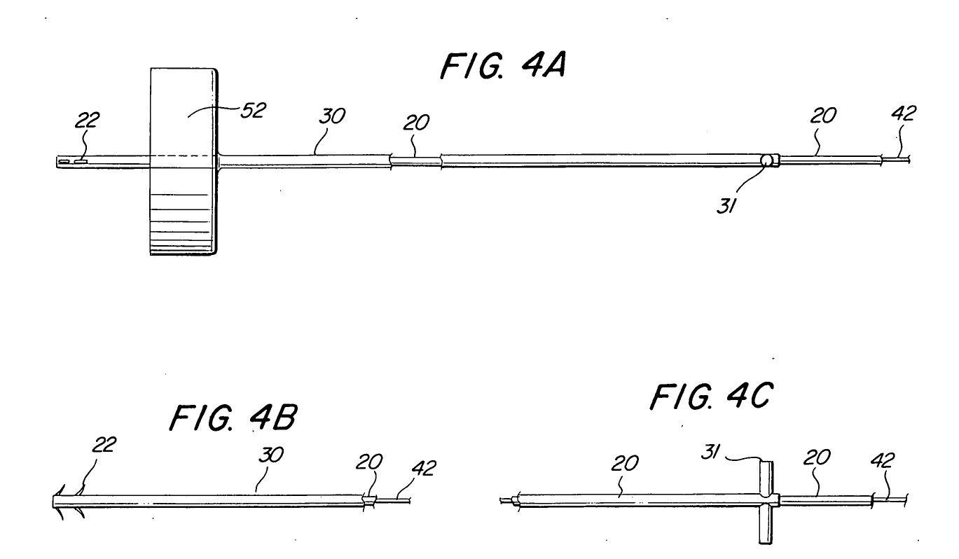

FIGS. 2A and 2B illustrate cross-sectional side views of the preferred embodiment of the present invention taken along sectional line 2-2 in FIG. 1A showing instrument 10 in an unengaged and engaged positions. The presently preferred cervical conization device 10 is initially shown in the unengaged position in FIG. 2A. Circular knife 18 is housed within the housing distal portion 12. The pentagonal knife blades 18a (FIG. 6C) surrounds a harpoon-like, barbed rod 20 that will ultimately attach to the patient's cervical tissue. In other words, the unengaged tool of FIG. 2A is inserted into the patient's vagina until the barbed 22 rod 20 pierces the tissue of a patient's cervix. Subsequently, the doctor squeezes the trigger handle 24 of the device 10 so that the blades 18a move to the engaged position of FIG. 2B. Note that the circular knife 18 is mounted to an end having an annulus 52 (FIG. 4A) of a hollow plunger bar 30 that slides back and forth, translationally with respect to a longitudinal axis 11 (see FIG. 2B). The hollow plunger 30 further slides back and forth along the barbed stabilization rod 20 when the handle 24 is opened and closed. As the handle 24 is squeezed, the hollow plunger 30 and pentagonal blades 18a carried in the annulus 52 connected to the hollow plunger 30 are pushed forward, out of the distal end of the device 10. Due to the angled or tapered annular shoulder 13 at the interior of the tool's housing distal portion 12, and further due to the geometry of the pentagonal blades 18a, the blades 18a are converged towards one another as they move outward in order to slice out a cone of biopsy tissue. The barbed 22 stabilization rod 20 prevents the cervical tissue from moving away as the blades 18a move outward from the device's 10 tapered annulus 13 and converge toward point 15. Once the blades 18a are fully converged, the doctor removes the device 10 from the patient along with the cervical cone biopsy sample. The doctor then disengages the device 10 and removes the biopsy tissue from the device for pathological examination.

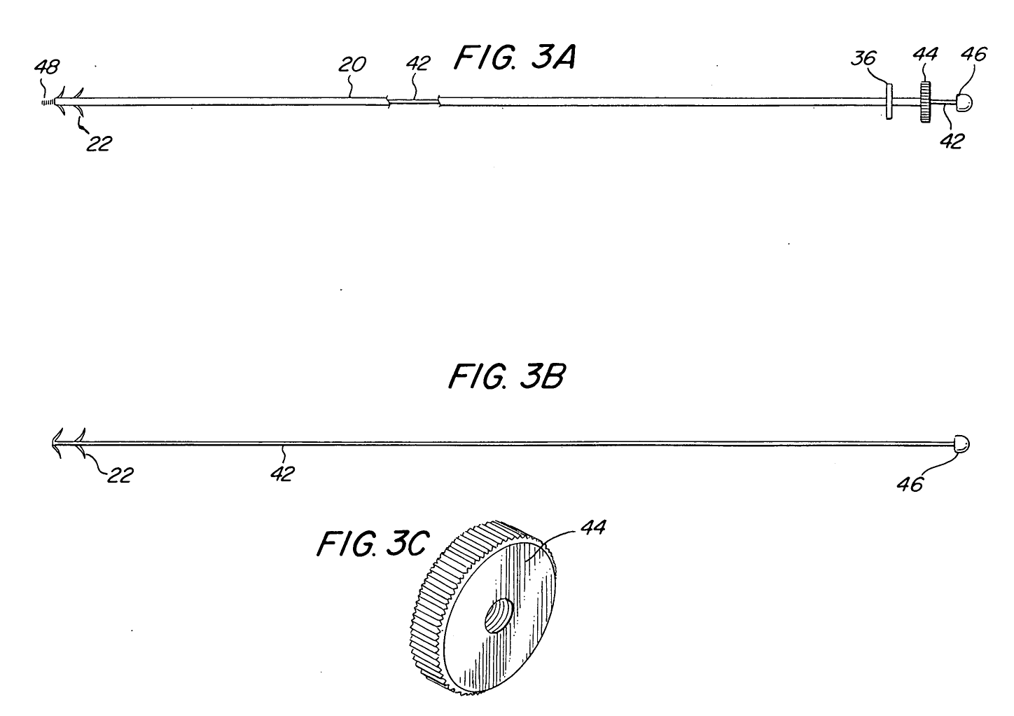

FIGS. 3A through 3C illustrate a preferred stabilization rod 20 of the present invention. In the embodiment shown, the stabilization rod 20 is comprised of an inner rod 42 and an outer sleeve 20. In the preferred embodiment, inner sleeve 42 is approximately 2 mm in diameter and outer sleeve 20 is approximately 3 mm in diameter, so that the rod 42 may move axially forward within the sleeve 43 against the bias of a spring 48 when knob 46 is depressed by the user's thumb. Outer sleeve 20 has the annular seat 36 that rests against the rear inside wall 17, discussed herein, and the annular seat 36 is opposed by nut 44 on the opposite side of the rear inside wall 17 (FIG. 2B). Nut 44 provides a means to adjust stabilization rod 20 with respect to device 10 before use. Inner rod 42 has a knob 46 at a proximal end that further provides means to adjust the position of the inner rod 42 and its flexible prongs 22 within the outer sleeve 43.

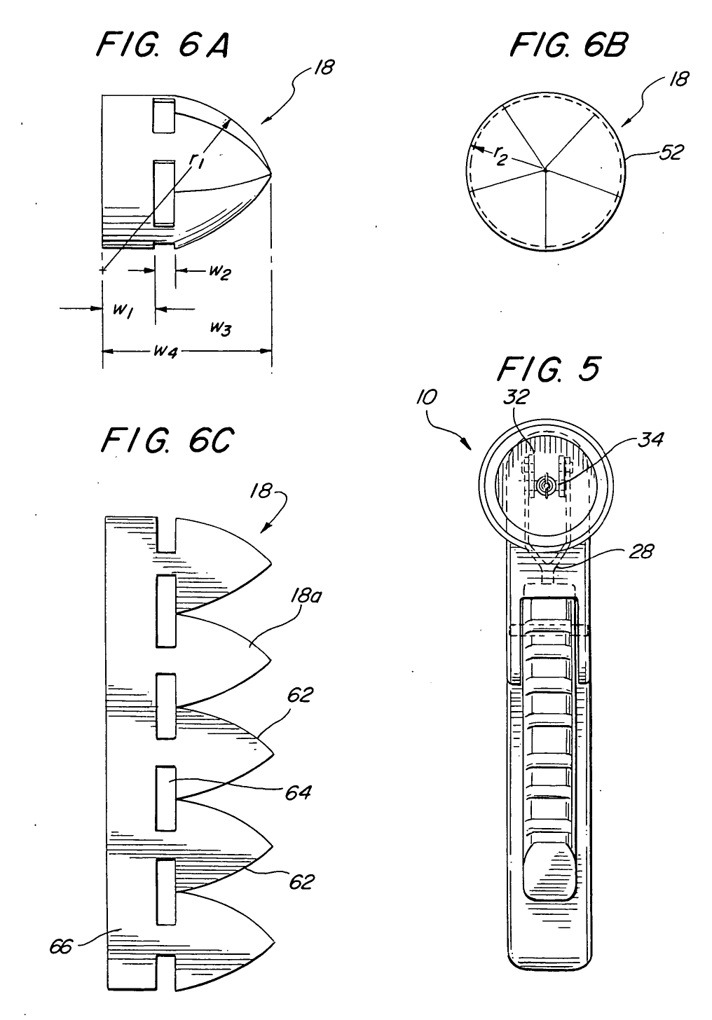

FIGS. 4A through 4C are side and top plan views of the hollow plunger 30 of the present invention further illustrating stabilization bar 20 with its barbs 22 threaded therethrough. The hollow plunger 30 has an annulus 52 for carrying the circular knife 18 according to a preferred embodiment of the invention. In a preferred embodiment, stabilization rod 20 has a diameter of approximately 3 mm while hollow plunger 30 has a diameter of approximately 5 mm so that the hollow plunger 30 may slide axially over the stabilization rod 20. FIG. 5 further illustrates an end view of the presently preferred embodiment of the present invention, illustrating the first and second pivot devices 32, 34.

Referring to FIGS. 6A through 6C a preferred circular pentagonal knife 18 of the present invention is further illustrated. Each blade 18a is doubled-edged 62 and converges against adjacent blades 18a during a cutting engagement to provide a clean cut. Radii r₁ and r₂ are constant to provide convergence upon a single point 15. In a preferred embodiment, r₁ is approximately equal to 1.5 cm and r₂ is approximately equal to 3.08 cm. Also according to the invention, r₂, the radius of the annulus 52, is selected according to the diameter of the cut desired. Similarly, the width dimensions w₁, w₂, w₃, w₄, particularly, w₃ precisely determine the depth of a tissue sample as surgical precision is a specific advantage of the present invention. It is further contemplated that the knife 18 of the invention is configured to be easily replaceable and disposable. In a preferred embodiment, w₁, w₂, w₃, w₄, are 10 mm, 4 mm, 19 mm and 33 mm, respectively. The diameter and depth of a particular tissue sample with vary with a particular patient is needed and may also depended on the size of any abnormality or lesion of interest.

Referring to FIG. 6C, a flat cut-out of circular knife 18 illustrated. Knife 18 may be stamped out of thin metal material known in the art and also has rectangular cut-outs 64 adjacent to an annulus width portion 66. The rectangular cut-outs 64 assist in enfolding the metal material about the annulus 52.

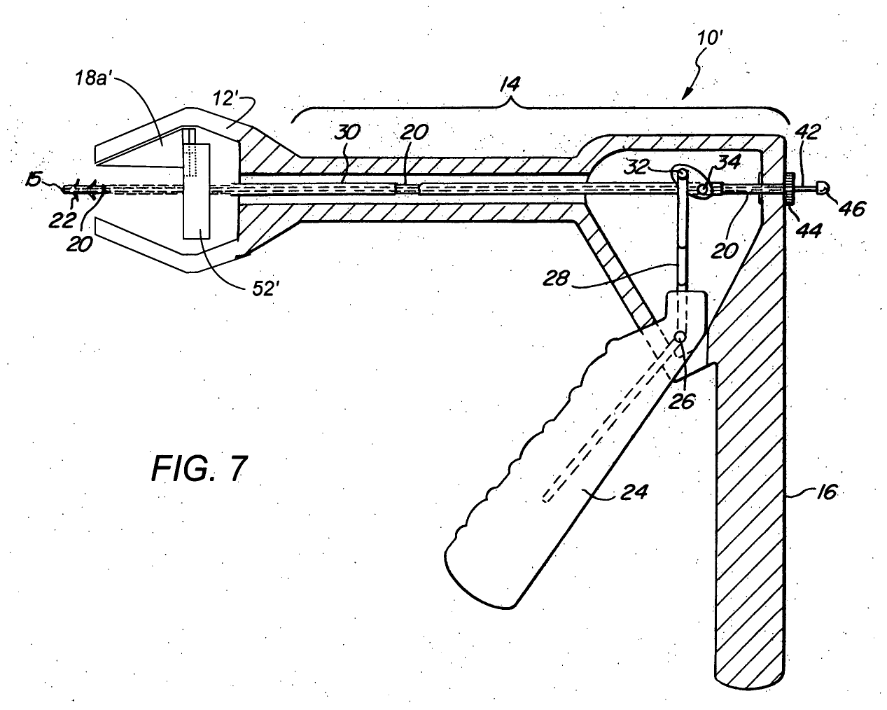

FIG. 7 is a cross-sectional view of a cervical conization device 10′ according to a second preferred embodiment of the invention. Here, the device 10! uses a tri-blade system where the three blades 18a′ move radially in and out within corresponding guide notches in an annulus 52′. As shown, the second preferred device 10′ features a distal portion 12′ that has been radially expanded relative to the first embodiment. The expanded geometry of the distal portion 12′ accommodates the radial movement of the blades 18a′ when the annulus 52′ is in a retracted position.

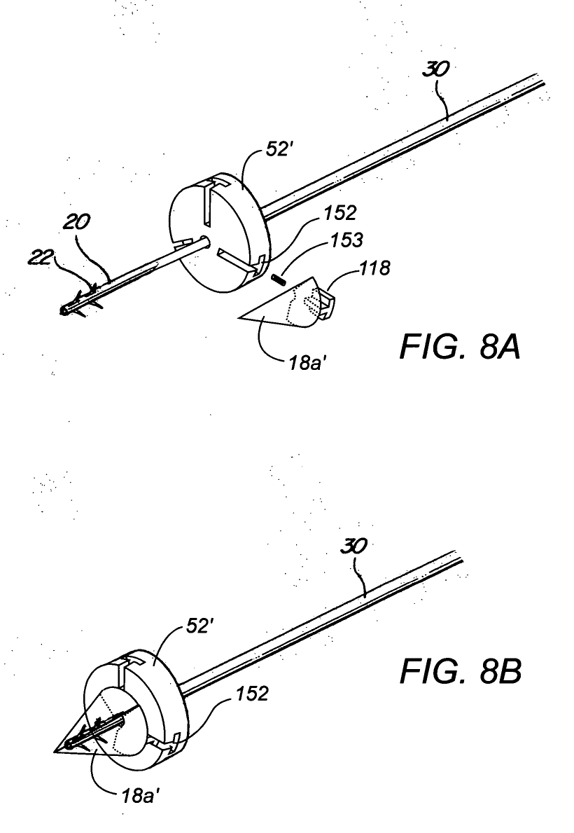

The operation of the second preferred cervical conization device 10′ is best understood with reference to FIGS. 8A and 8B. FIG. 8A, in particular, is an exploded perspective view of a portion of the second preferred embodiment shown in FIG. 7, showing how a guide tab 118 on the bottom of one of the three blades 18a′ engages the guide notch 152 in the annulus 52′, a spring 153 being present to bias the blade 18a′ outward. FIG. 8B shows the position of all three blades 18a′ when the annulus 52′ in the assembly suggested by FIGS. 7 and 8A is moved forward and the blades are converged by the geometry of the distal portion 12′ (see FIG. 7).

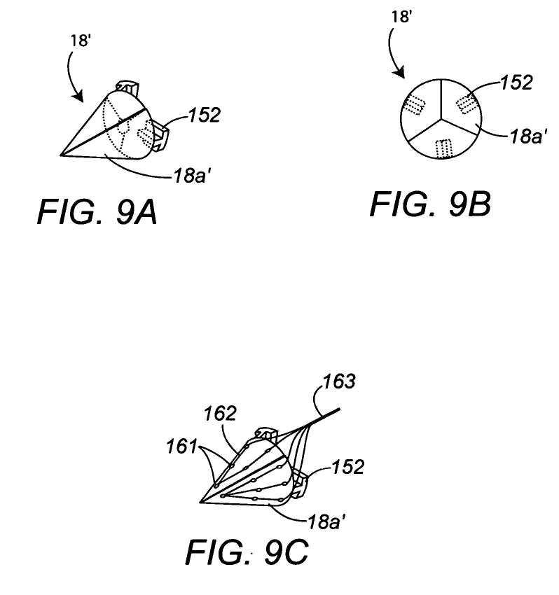

FIG. 9A is a perspective view of the second preferred blade assembly 18′ and FIG. 9B is an end view of the second preferred blade assembly 18′. As shown, the blades 18a′ are only curved about the long axis. The guide tabs 152 are mounted in a suitable fashion to a base (not separate numbered) of each blade 18a′ . The guide tabs 152 are made of a rigid, low friction material such as Teflon or UHMW plastic.

It is believed that this second preferred cervical conization device 10′ will provided the added benefit of preventing the blades from bending or “troughing”, and failing to converge on a common point, when deployed into the patient's tissue.

FIG. 9C is a perspective view of the second preferred blade assembly 18′ with resistive pads 161, conductors 162, and a wire bundle 163 for implementing an electro-cauterization process on the exterior of the second preferred blade assembly 18′. The wire bundle 163, of course, leads to a suitable cautering device that drives current through the resistive pads 161 and creates sufficient heat to cauterize the biopsy bed, i.e. the tissue adjacent to the biopsy site. Because it is desirable to avoid cauterizing the biopsy tissue itself, the interior of the blades 18a should be coated with a suitable material that provides sufficient thermal insulation.

Many alterations and modifications may be made by those having ordinary skill in the art without departing from the spirit and scope of the invention. Therefore, it must be understood that the illustrated embodiments have been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

While the particular Cervical Conization Device as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

1. A surgical instrument for excising a tissue sample from a tissue mass, the surgical instrument comprising:
a hollow plunger bar defining a longitudinal axis having a distal and proximal end;
a circular knife having a plurality of blades converging on a point of convergence, the circular knife disposed about the distal end of the hollow plunger bar, the circular knife further having engaged and unengaged positions; and
a housing having a distal portion accommodating the circular knife, the housing distal portion having an annulus tapered at an end thereof, the tapered annulus directing the circular knife toward the point of convergence.

2. The surgical instrument of claim 1, further comprising a stabilization rod disposed inside of the hollow plunger bar, the stabilization rod further having a distal and a proximal end.

3. The surgical instrument of claim 1, the housing further comprising:
an elongated sleeve portion about the longitudinal axis and housing the stabilization rod and the hollow plunger bar; and
a stationary handle portion extending substantially perpendicular to the elongated sleeve portion.

4. The surgical instrument of claim 3, further comprising a trigger handle pivotally mounted opposing the stationary handle portion, the trigger handle for actuating the hollow plunger bar.

5. The surgical instrument of claim 4, further comprising:
a fulcrum pin for pivotally mounting the trigger handle;
a Y-shaped swing arm connected to the fulcrum pin opposite the trigger handle; and
a first pivot device and a second pivot device, the first pivot device connected to the Y-shaped swing arm and the second pivot device connected to the hollow plunger bar, the first and second pivot devices together for converting rotational movement of the Y-shaped swing arm to translational movement of the hollow plunger bar.

6. The surgical instrument of claim 1, the hollow plunger bar having a tee on the proximal end.

7. The surgical instrument of claim 5, the hollow plunger bar having a tee on a proximal end, the tee having first and second ends, wherein the Y-shaped swing arm having two portions each connecting to one of two pins of the first pivot device, each of the two pins of the first pivot device connecting to a segment connecting to each of two pins of the second pivot device, the each of two pins connected to the tee first and second ends respectively.

8. The surgical instrument of claim 3, the housing elongated sleeve portion having a rear inside wall at a proximal end, the stabilization rod having a seat securing the stabilization rod against the rear inside wall, the housing further having a rear outside wall opposing the rear inside wall, the stabilization rod further comprising a nut receiving a threaded portion of the stabilization rod at the rear outside wall, the nut for adjusting a relative position of the stabilization rod with respect to the housing.

9. The surgical instrument of claim 2, the stabilization rod having a spring secured to the distal end.

10. The surgical instrument of claim 2, the stabilization rod having an outer sleeve disposed about an inner rod, the inner rod having flexible barbs secured to the distal end, the flexible barbs for attaching to cervical tissue, wherein the flexible barbs can further be retracted and housed inside the outer sleeve.

11. The surgical instrument of claim 1, wherein the circular knife is a pentagonal knife having five blades converging on the point of convergence.

12. The surgical instrument of claim 1, wherein the circular knife plurality of blades each have two curved edges converging at a point, the edges having a constant radius of curvature.

13. The surgical instrument of claim 12 wherein the constant radius of curvature of the edges is approximately equal to 3.08 centimeters.

14. The surgical instrument of claim 1, wherein the circular knife comprises a flat cut-out composed of metal material and enfolded about an annulus, the annulus having a radius.

15. The surgical instrument of claim 13, wherein the circular knife annulus radius is approximately equal to 1.50 centimeters.

16. The surgical instrument of claim 14, wherein the circular knife annulus has a width, the width approximately equal to 1.0 centimeters.

17. The surgical instrument of claim 14, wherein the circular knife flat cut-out comprises a rectangular cut-out to assist in enfolding the metal material about the annulus.

18. A surgical instrument for excising a tissue sample from a tissue mass, the surgical instrument comprising:
a stabilization rod having a distal and a proximal end and defining a longitudinal axis;
a hollow plunger bar disposed about the stabilization rod having a distal and proximal end;
a circular knife having a plurality of blades converging on a point of convergence, the circular knife disposed about the distal end of the hollow plunger bar, the circular knife further having engaged and unengaged positions; and
a housing having a distal portion accommodating the circular knife, the housing distal portion having an annulus tapered at an end thereof, the tapered annulus directing the circular knife toward the point of convergence.

19. The surgical instrument of claim 18, the housing further comprising:
an elongated sleeve portion about the longitudinal axis and housing the stabilization rod and the hollow plunger bar; and
a stationary handle portion extending substantially perpendicular to the elongated sleeve portion.

20. The surgical instrument of claim 19, further comprising a trigger handle pivotally mounted opposing the stationary handle portion, the trigger handle for actuating the hollow plunger bar.

21. The surgical instrument of claim 20, further comprising:
a fulcrum pin for pivotally mounting the trigger handle;
a Y-shaped swing arm connected to the fulcrum pin opposite the trigger handle; and
a first pivot device and a second pivot device, the first pivot device connected to the Y-shaped swing arm and the second pivot device connected to the hollow plunger bar, the first and second pivot devices together for converting rotational movement of the Y-shaped swing arm to translational movement of the hollow plunger bar.

22. The surgical instrument of claim 18, the hollow plunger bar having a tee on the proximal end.

23. The surgical instrument of claim 21, the hollow plunger bar having a tee on a proximal end, the tee having first and second ends, wherein the Y-shaped swing arm having two portions each connecting to one of two pins of the first pivot device, each of the two pins of the first pivot device connecting to a segment connecting to each of two pins of the second pivot device, the each of two pins connected to the tee first and second ends respectively.

24. The surgical instrument of claim 19, the housing elongated sleeve portion having a rear inside wall at a proximal end, the stabilization rod having a seat securing the stabilization rod against the rear inside wall, the housing further having a rear outside wall opposing the rear inside wall, the stabilization rod further comprising a nut receiving a threaded portion of the stabilization rod at the rear outside wall, the nut for adjusting a relative position of the stabilization rod with respect to the housing.

25. The surgical instrument of claim 18 the stabilization rod having a spring secured to the distal end.

26. The surgical instrument of claim 18 the stabilization rod having an outer sleeve disposed about an inner rod, the inner rod having flexible barbs secured to the distal end, the flexible barbs for attaching to cervical tissue, wherein the flexible barbs are further able to be retracted and housed inside the outer sleeve.

27. The surgical instrument of claim 18, wherein the circular knife is a pentagonal knife having five blades converging on the point of convergence.

28. The surgical instrument of claim 18, wherein the circular knife plurality of blades each have two curved edges converging at a point, the edges having a constant radius of curvature.

29. The surgical instrument of claim 28 wherein the constant radius of curvature of the edges is approximately equal to 3.08 centimeters.

30. The surgical instrument of claim 18, wherein the circular knife comprises a flat cut-out composed of metal material and enfolded about an annulus, the annulus having a radius.

31. The surgical instrument of claim 30, wherein the circular knife annulus radius is approximately equal to 1.50 centimeters.

32. The surgical instrument of claim 30, wherein the circular knife annulus has a width, the width approximately equal to 1.0 centimeters.

33. The surgical instrument of claim 30, wherein the circular knife flat cut-out comprises a rectangular cut-out to assist in enfolding the metal material about the annulus.

34. A surgical instrument for excising a tissue sample from a tissue mass, the surgical instrument comprising:
a hollow plunger bar disposed about the stabilization rod having a distal and proximal end; and
a circular pentagonal knife having five blades converging on a point of convergence, the circular knife disposed about the distal end of the hollow plunger bar, the circular knife further having engaged and unengaged positions.

35. The surgical instrument of claim 34, further comprising:
a housing having a distal portion accommodating the circular knife, the housing distal portion having an annulus tapered at an end thereof, the tapered annulus directing the circular knife toward the point of convergence; and
a stabilization rod having a distal and a proximal end and defining a longitudinal axis.

36. The surgical instrument of claim 35 the housing further comprising:
an elongated sleeve portion about the longitudinal axis and housing the stabilization rod and the hollow plunger bar; and
a stationary handle portion extending substantially perpendicular to the elongated sleeve portion.

37. The surgical instrument of claim 36, further comprising a trigger handle pivotally mounted opposing the stationary handle portion, the trigger handle for actuating the hollow plunger bar.

38. The surgical instrument of claim 37, further comprising:
a fulcrum pin for pivotally mounting the trigger handle;
a Y-shaped swing arm connected to the fulcrum pin opposite the trigger handle; and
a first pivot device and a second pivot device, the first pivot device connected to the Y-shaped swing arm and the second pivot device connected to the hollow plunger bar, the first and second pivot devices together for converting rotational movement of the Y-shaped swing arm to translational movement of the hollow plunger bar.

39. The surgical instrument of claim 34, the hollow plunger bar having a tee on the proximal end.

40. The surgical instrument of claim 38 the hollow plunger bar having a tee on a proximal end, the tee having first and second ends, wherein the Y-shaped swing arm having two portions each connecting to one of two pins of the first pivot device, each of the two pins of the first pivot device connecting to a segment connecting to each of two pins of the second pivot device, the each of two pins connected to the tee first and second ends respectively.

41. The surgical instrument of claim 36, the housing elongated sleeve portion having a rear inside wall at a proximal end, the stabilization rod having a seat securing the stabilization rod against the rear inside wall, the housing further having a rear outside wall opposing the rear inside wall, the stabilization rod further comprising a nut receiving a threaded portion of the stabilization rod at the rear outside wall, the nut for adjusting a relative position of the stabilization rod with respect to the housing.

42. The surgical instrument of claim 35, the stabilization rod having a spring secured to the distal end.

43. The surgical instrument of claim 35, the stabilization rod having an outer sleeve disposed about an inner rod, the inner rod having flexible barbs secured to the distal end, the flexible barbs for attaching to cervical tissue, wherein the flexible barbs are further able to be retracted and housed inside the outer sleeve.

44. The surgical instrument of claim 44 wherein the five blades each have two curved edges converging at a point, the edges having a constant radius of curvature.

45. The surgical instrument of claim 44 wherein the constant radius of curvature of the edges is approximately equal to 3.08 centimeters.

46. The surgical instrument of claim 34, wherein the circular knife comprises a flat cut-out composed of metal material and enfolded about an annulus, the annulus having a radius.

47. The surgical instrument of claim 46, wherein the circular knife annulus radius is approximately equal to 1.50 centimeters.

48. The surgical instrument of claim 46, wherein the circular knife annulus has a width, the width approximately equal to 1.0 centimeters.

49. The surgical instrument of claim 46, wherein the circular knife flat cut-out comprises a rectangular cut-out to assist in enfolding the metal material about the annulus.
